(19) United States
(12) Patent Application Publication
(10) Pub. No.: US 2005/0273054 A1
(43) Pub. Date: Dec. 8, 2005

(54) Epinephrine auto-injector

(75) Inventor: Lee Ann Asch, Boca Raton, FL (US)

Correspondence Address: AKERMAN SENTERFITT, P.O. BOX 3188, WEST PALM BEACH, FL, 33402-3188, US

(73) Assignee: Florida Atlantic University, Boca Raton, FL (US)

(21) Appl. No.: 11/143,879

(22) Filed: Jun. 2, 2005

Related U.S. Application Data
(60) Provisional application No. 60/576,636, filed on Jun. 3, 2004.

(51) Int. Cl. A61M 5/20

(52) U.S. Cl. 604/136; 604/198

(57) ABSTRACT

An epinephrine auto-injector that is constructed and arranged to reduce the likelihood of accidental discharge of the injector The epinephrine injector may also be constructed and arranged to be safer to use and/or dispose of after use. As such, the present invention provides a epinephrine injector that may include a locking mechanism, an actuation mechanism, or both, to help prevent the accidental discharge of the injector. In addition, the epinephrine injector may include a two-part housing and may include means for covering the needle after the epinephrine injector has been used to help dispose of the epinephrine injector after use.

### CROSS REFERENCE TO RELATED APPLICATIONS

This application claims priority to U.S. Provisional Patent Application No. 60/576,636, which was filed Jun. 3, 2004.

### FIELD OF THE INVENTION

The present invention is directed to drug injection systems. In particular, the present invention is directed to epinephrine injection systems.

### BACKGROUND OF THE INVENTION

Certain individuals are highly allergic to foods, including peanuts, other nuts, wheat, milk and shellfish. Others are highly allergic to the stings of insects, such as bees and wasps, to latex and/or to medications. When allergic patients are exposed to these allergens, a severe reaction may occur (anaphylaxis) which may be life-threatening if not treated immediately.

Epinephrine is generally used to treat an allergic patient at the onset of an anaphylactic reaction. Epinephrine quickly relieves bronchial swelling, constricts blood vessels, relaxes smooth muscles in the lungs, stimulates the heartbeat, and acts to reverse swelling, thus allowing the patient to function until further treatment can be obtained.

Because exposure is unpredictable, the reaction may occur quickly, and the patient may not be near medical help at the time of exposure, patients who are subject to severe anaphylactic reactions must carry epinephrine at all times. It is also necessary that the patient be able to self-administer the epinephrine while experiencing an allergic reaction. To address this need, epinephrine is generally prescribed in an auto-injector, e.g., a device commercially available under the tradename “EpiPen”. The auto-injector has a spring-activated, concealed needle that, when triggered, springs forward to deliver a dose of epinephrine.

While epinephrine auto-injectors are life-saving devices when properly cared for and used, a number of safety precautions should be observed with these devices.

It is important that the auto-injector be used only for intramuscular (rather than intravenous) injection. Currently, manufacturers generally instruct that the auto-injector be used only on the patient's thigh. Injection of epinephrine into other areas can be dangerous. For example, injection into an extremity, such as a hand or foot, can shut off blood supply to that area, resulting in potential damage to the extremity. Vascular injection may also be potentially dangerous because systemic delivery of the epinephrine may cause complications in some patients due to a sharp rise in blood pressure produced by the epinephrine.

Jostling and bumping of the auto-injector while the patient is carrying the auto-injector has been known to cause accidental triggering of the spring mechanism. Accidental triggering of the auto-injector can result in injury to the patient or a caregiver or bystander. Also, accidental triggering may exhaust the epinephrine in the auto-injector, so that the epinephrine is not available when needed during an anaphylactic reaction.

The patient may suffer extreme illness and even death if the auto-injector is not in useable condition when it is needed. For example, the patient may not be able to self-inject the epinephrine if the spring-activation mechanism has been damaged by jostling or impact of the auto-injector during storage and transport by the patient.

The patient is also in danger if the medicine contained in the injector has deteriorated. Epinephrine is heat and light-sensitive, and as a result if the auto-injector is exposed to direct sunlight or extreme heat the epinephrine may oxidize, potentially rendering it ineffective. Epinephrine turns brown when oxidized, so auto-injectors are typically provided with a window to allow the patient to regularly inspect the color of the medication.

Accordingly, what is needed is an auto-injector that is designed to help prevent the auto-injector from accidental triggering, damage due to jostling or impact, and/or prolonged periods of exposure to light. It would also be beneficial to provide an auto-injector that the patient or a caregiver (potentially a small child or elderly family member) is able to use easily and quickly in a crisis situation.

### SUMMARY OF THE INVENTION

The present invention is an epinephrine injector. The epinephrine injector is constructed and arranged to reduce the likelihood of accidental discharge of the injector such that it is more likely to be available when needed. In alternative embodiments, the epinephrine injector is constructed and arranged to be safer to use and/or dispose of after use.

As such, the present invention provides an epinephrine injector that may include a locking mechanism, an actuation mechanism, or both, to help prevent the accidental discharge of the injector. In addition, in other embodiments, the epinephrine injector uses a two-part housing, thereby reducing the size of the device and/or reducing the risk of accidental discharge. In still other embodiments, the epinephrine injector may include means for covering the needle after the epinephrine injector has been used to help dispose of the epinephrine injector after use.

In particular, the present invention provides an epinephrine injector including a housing having a first housing and a second housing; a needle; a source of epinephrine; and a spring for causing the needle to inject the epinephrine; wherein the first housing is movable relative to the second housing such that movement of the first housing relative to the second housing permits the spring to release, thereby causing injection of the epinephrine by the needle.

### DETAILED DESCRIPTION OF THE DRAWINGS

Other objects, features and advantages of the present invention will become apparent upon reading the following detailed description, while referring to the attached drawings, in which:

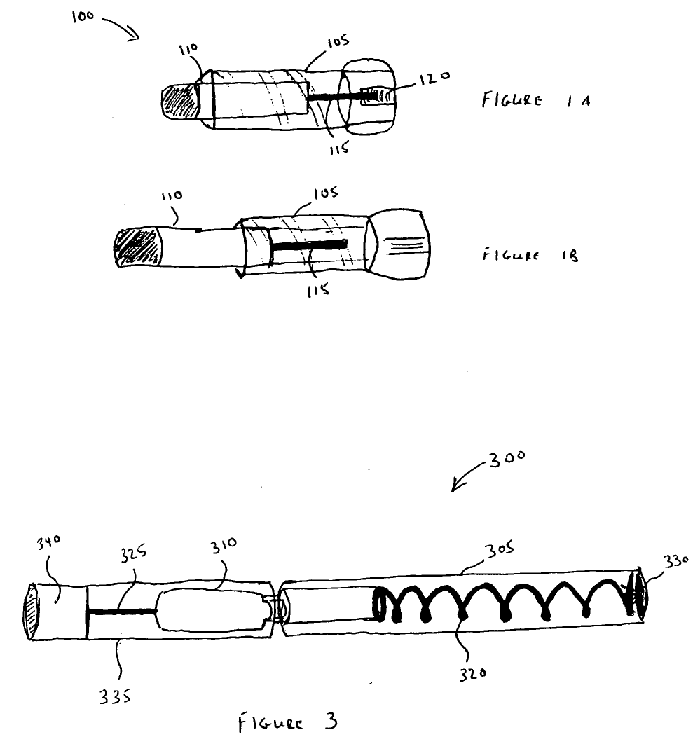

FIGS. 1A and 1B are a general depiction of an epinephrine injector according to one embodiment of the present invention in A) a locked position and B) an unlocked position.

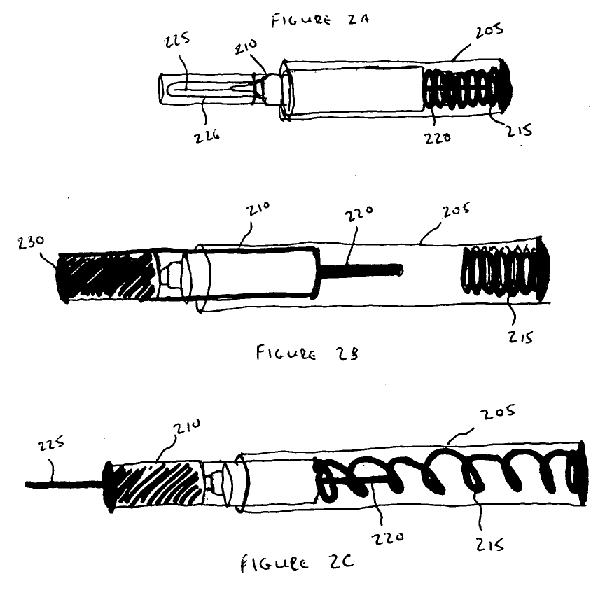

FIGS. 2A, 2B and 2C are a detailed depiction of an epinephrine injector according to one embodiment of the present invention in A) a locked position; B) an unlocked position and unfired position; and C) a fired position.

FIG. 3 is a detailed depiction of an epinephrine injector according to another embodiment of the present invention.

### DETAILED DESCRIPTION OF THE INVENTION

The present invention is more particularly described in the following examples that are intended to be illustrative only since numerous modifications and variations therein will be apparent to those skilled in the art. As used in the specification and in the claims, the singular form “a,““an,” and “the” may include plural referents unless the context clearly dictates otherwise. Also, as used in the specification and in the claims, the term “comprising” may include the embodiments “consisting of” and “consisting essentially of.”

The present invention provides an epinephrine injector that alleviates one or more of the problems associated with prior art epinephrine pens. In the past, epinephrine pens would generally include a tubular housing containing the epinephrine to be injected. At one end was a locking mechanism that, when removed, unlocked a spring. Once the spring was unlocked, pressure on the tip at the other end would cause the spring to release, thereby forcing a needle to be forced from the housing and into the person's muscle, thereby injecting the epinephrine. Once used, the device would leave an exposed needle. Also, if the locking mechanism were accidentally knocked off during day-to-day movements (as these epinephrine pens may be carried in purses, pockets and/or pouches), the needle may accidentally be released such that the pen would not be available in an emergency, or even possibly injecting the carrier by accident.

The present invention provides an epinephrine injector that includes one or more safety mechanisms for helping to prevent accidental discharge of the injector and/or increase safety after the epinephrine injector has been used. In a first embodiment, the present invention provides an epinephrine injector having a housing having a needle connected to a source of epinephrine therein. The housing also includes a spring for forcing the needle from the housing when the spring or other mechanism is actuated, thereby permitting the source of epinephrine to be injected into the individual. As used herein, a “spring” is a spring or any other related mechanism that is capable of causing a needle to be ejected from a housing such that epinephrine may be injected into an individual through the needle. In an alternative embodiment, the “spring” may be a pressurized gas cartridge. As used herein, a “needle” is a needle or any other mechanism capable of injecting epinephrine into an individual.

The present invention also includes an actuation mechanism for actuating the spring such that the spring causes the needle to exit the housing once the actuation mechanism has been activated. As accidental actuation may occur, it is beneficial for the spring to be locked or otherwise prohibited from releasing the needle until such time as it is needed. In one embodiment, the actuation mechanism is a pressure-sensitive release that activates once a sufficient amount of pressure has been applied to the release.

As such, the present invention also includes a locking mechanism for locking the spring such that the spring does not accidentally actuate. This locking mechanism may, in one embodiment, be located at a first end of the epinephrine injector such that, when actuated, the needle exits a second end of the housing.

The locking mechanism engages the spring that is used to force the needle out of the housing and into the individual to be injected. Once the locking mechanism has been released or otherwise disengaged from the spring, the spring is now free to be actuated. However, if the locking mechanism has not been released or otherwise disengaged from the spring, the spring is unable to actuate, even if the actuation mechanism is activated, or is attempted to be activated. As such, in an embodiment wherein the actuation mechanism is a pressure-sensitive release, application of pressure to the actuation mechanism would not cause the spring to release if the locking mechanism has not been released or otherwise disengaged. In one embodiment, the locking mechanism is a pin that, when removed, permits the spring to release.

While, in some embodiments, it may be beneficial for the spring to release once the locking mechanism has been released or otherwise disengaged from the spring, it is contemplated that, in other embodiments, the spring will not release when the locking mechanism is removed. Rather, the spring will only release once the actuation mechanism is activated. As such, even if the locking mechanism is accidentally disengaged, the spring would not release until the actuation mechanism was activated by pressure.

Alternatively, in other embodiments, the present invention does not utilize an actuation mechanism, but only uses a locking mechanism. As such, in this embodiment, release of the locking mechanism would cause the spring to automatically release. This embodiment may be used for those individuals that may have difficulty activating the actuating mechanism.

In one embodiment, the present invention uses a housing having two parts. The first housing would be an external shell or portion. The second housing would be an internal portion that is contained, at least partially, within the first housing and that is movable relative to the first housing. As such, the second housing may be moved such that a greater or a lesser amount of the second housing was contained in, or external to, the first housing portion. In one embodiment, the needle, spring and epinephrine would be located in the second housing portion, while the locking mechanism would be located in the first housing portion, but wherein the locking mechanism was capable of engaging and locking/unlocking the spring. In another embodiment, the needle, spring and epinephrine would be located in the first housing portion, while the locking mechanism would be located in the second housing portion, but wherein the locking mechanism was capable of engaging and locking/unlocking the spring. In other embodiments, the needle, epinephrine and locking mechanism are in one housing and the spring is in the other housing although, again, the locking mechanism would still be capable of engaging and locking/unlocking the spring.

As such, in the embodiments using a two-part housing, it would be contemplated that, in a locked position, a greater portion of the second housing would be contained within the first housing, while in an unlocked position, it would be contemplated that a lesser portion of the second housing would be contained within the first housing.

The second housing may be movable relative to the first housing such that movement of the second housing would cause the locking mechanism to release or otherwise disengage the spring. In this embodiment, the second housing may be moved in any manner relative to the first housing as long as the locking mechanism were caused to release or otherwise disengage the spring. In one embodiment, the second housing may be slidable relative to the first housing such that sliding the second housing away from the first housing, or sliding the first housing away from the second housing, would cause the locking mechanism to release or otherwise disengage the spring. In another embodiment, the second housing may be rotatable relative to the first housing such that rotating either the first housing or the second housing would cause the locking mechanism to release or otherwise disengage the spring.

By using a two-part housing, it is possible to reduce the amount of space the epinephrine injector would occupy, thereby permitting smaller epinephrine injectors to be used, unlike current epinephrine pens. Additionally, by permitting the two parts to move relative to each other, it is possible to reduce the chance of the locking mechanism being knocked off or otherwise accidentally released.

The housing may be made from any material capable of containing the needle, spring and epinephrine. In beneficial embodiments, the housing is made from a material that is opaque or substantially opaque as this material does not permit exposure of the epinephrine to light, which may reduce or eliminate the effectiveness of the epinephrine. If a two part housing is used, one or both parts of the housing may be opaque or substantially opaque. In select embodiments, the housing is plastic.

In an alternative embodiment, the present invention does not use a locking mechanism. Rather, the epinephrine injector would include a cover or cap or other means for covering the actuation mechanism. In this embodiment, the spring would be unlocked, but would not release until the actuation mechanism was activated. The cover would prevent this result from occurring.

Even if a locking mechanism were used, another embodiment of the present invention may also include a cover for the device. In this embodiment, a piece of cork or similar material may be attached to an inner portion of the cover. As such, in this embodiment, once the epinephrine injector has been used, the cover may be placed on the spent injector with the cork or similar material covering the tip of the needle. As such, the cork or similar material helps to prevent someone from accidentally getting stuck by the needle and permits safe disposal of the epinephrine injector after use.

Various additional features may be incorporated into the epinephrine injector of the present invention through various alternative embodiments. For example, in one embodiment, a window or other means may be placed in one or more parts of the housing to permit an individual to view the epinephrine to ensure that it is still present and/or has not expired. In an alternative embodiment, an expiration date may be provided for the epinephrine on one end of the housing or in another suitable location. In still other embodiments, means for carrying the epinephrine injector may also be provided. The means may include, but are not limited to, key rings, hooks, eyelets for attaching to a hook or a cord, and a combination thereof.

The present invention will now be further described through reference to various embodiments. It is to be understood that these embodiments are non-limiting and are presented to provide a better understanding of the present invention.

FIGS. 1A and 1B provide a locked (FIG. 1A) and unlocked (FIG. 1B) depiction of one embodiment of an epinephrine injector of the present invention. As seen in FIGS. 1A and 1B, the epinephrine injector 100 includes a housing having a first housing 105 and a second housing 110. In the locked position, a substantial portion of the second housing 110 is contained within the first housing 105. In the unlocked position, a smaller portion of the second housing 110 is contained within the first housing 105. In this embodiment, the first housing 105 is constructed from a substantially transparent material while the second housing 110 is constructed from a substantially opaque material.

The epinephrine injector 100 also includes a locking mechanism 115 on the second housing 105 that is connected to the spring 120. When the epinephrine injector 100 is unlocked, the locking mechanism 115 disengages from the spring 120, thereby permitting the spring to be released. The needle and epinephrine (both not shown) are contained within second housing 110.

FIGS. 2A, 2B and 2C provide a locked (FIG. 2A), unlocked and unfired (FIG. 2B) and fired (FIG. 2C) depiction of one embodiment of an epinephrine injector of the present invention. As seen in FIGS. 2A and 2B, the epinephrine injector 200 includes a housing having a first housing 205 and a second housing 210. As with FIGS. 1A and 1B, in the locked position, a substantial portion of the second housing 210 is contained within the first housing 205. In the unlocked position, a smaller portion of the second housing 210 is contained within the first housing 205.

The epinephrine injector 200 also includes a locking mechanism 215 in the first housing 205 that is connected to a portion of the spring 220. The needle 225 and epinephrine 226 are in the second housing 210. As seen in FIGS. 2B and 2C, the spring 220 remains locked until the spring 220 is actuated, at which time the spring 220 forces the needle 225 from the second housing. The spring 220 may be actuated by an actuation mechanism 230 at one end of the second housing 210.

FIG. 3 provides another embodiment of an epinephrine injector 300 and having a first housing 305, a second housing 310, a spring 320, an actuation mechanism 330 at one end of the first housing wherein depression of the actuation mechanism 330 causes spring 320 to release and firing needle 325 from the second housing 310. This embodiment also include a cover 335 having a piece of cork 340 therein such that, after the epinephrine injector 300 has been used, the cover 335 may be placed on the epinephrine injector 300 and the cork 340 may cover the tip of the needle 325 such that the needle 325 cannot accidentally stick someone, thereby making the epinephrine injector 300 safer to use.

Although the illustrative embodiments of the present disclosure have been described herein with reference to the accompanying drawings and examples, it is to be understood that the disclosure is not limited to those precise embodiments, and various other changes and modifications may be affected therein by one skilled in the art without departing from the scope of spirit of the disclosure. All such changes and modifications are intended to be included within the scope of the disclosure as defined by the appended claims.

1. An epinephrine injector comprising:
a housing comprising a first housing and a second housing;
a needle;
a source of epinephrine; and
a spring for causing the needle to inject the epinephrine;
wherein the first housing is movable relative to the second housing such that movement of the first housing relative to the second housing permits the spring to release, thereby causing injection of the epinephrine by the needle.

2. The epinephrine injector of claim 1, wherein the first housing is movable relative to the second housing by rotating the first housing relative to the second housing.

3. The epinephrine injector of claim 1, wherein the first housing is movable relative to the second housing by sliding the first housing relative to the second housing.

4. The epinephrine injector of claim 1, further comprising a locking mechanism for locking the spring such that the spring is not capable of releasing until the locking mechanism is disengaged.

5. The epinephrine injector of claim 1, further comprising an actuation mechanism for releasing the spring such that the spring does release until the actuation mechanism is activated.

6. The epinephrine injector of claim 1, further comprising a locking mechanism for locking the spring such that the spring is not capable of releasing until the locking mechanism is disengaged and an actuation mechanism for releasing the spring such that the spring does release until the actuation mechanism is activated.

7. The epinephrine injector of claim 1, further comprising a cover for covering at least a portion of the housing.

8. The epinephrine injector of claim 7, further comprising cork attached to an inner surface of the cover.
